(19) United States
(12) Patent Application Publication
(10) Pub. No.: US 2008/0306551 A1
(43) Pub. Date: Dec. 11, 2008

(54) Surgical Fastening

(75) Inventors: Marc Sanders, Overijssel (NL); Paul Firkins, Neuchatel (CH)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: DEPUY INTERNATIONAL LTD, Leeds (GB)

(21) Appl. No.: 10/599,209

(22) PCT Filed: Mar. 24, 2005
(86) PCT No.: PCT/GB2005/001170

§ 371 Date: Aug. 28, 2008

(30) Foreign Application Priority Data
Mar. 25, 2004 (GB) 0406822.7

(51) Int. Cl. A61B 17/04; A61B 17/70

(52) U.S. Cl. 606/301; 606/246; 606/264

(57) ABSTRACT

A surgical fastening (160), kit of parts including the fastening and method for stabilising a vertebra using the fastening are described. The fastening is used to attach a rod (166) to a spinal body part. The fastening comprises a split ring (160) of a material which defines a channel for receiving the rod. A formation can receive a fixing (168) to secure the fastening to the spinal body part. A closure mechanism is operable to close the ring. The material is sufficiently flexible to allow the fastening to be closed about the rod at least once, without weakening.

The present invention relates to a fastening, and in particular to a fastening for use in spinal surgical procedures for securing a spinal body part to a support rod.

For some patient conditions it can be necessary to provide a rigid support structure for the back so as to immobilise some or all of the vertebrae. One way of doing this is by use of rigid rods, which can be curved in order to more closely match the shape of the spine in the region to be supported. The rod, or rods if one is positioned on either side of the spine, are attached to the vertebrae and other body parts by various fixings, such as hooks.

Typically the surgeon wants to be able to attach vertebrae to the rod or rods at many positions along their length so as to ensure that the vertebrae are correctly supported and immobilised. This means that a large number of fixing or fastening devices need to be ‘pre-loaded’ onto the rod by being threaded onto the rod. However, if it subsequently transpires, with the rod in situ attached to the spine, that a fixing is incorrect or that a different fixing would be appropriate, then the rod has to be dismounted in order to change the fixing. Further, it is very difficult to manipulate and handle the rod when attaching it to the spine as all the fixings can get in the way when trying to initially mount the rod.

Hence, although this technique can be of great therapeutic value to patients, there can be a tendency amongst surgeons not to use it, owing to the practical difficulties.

Therefore, there is a need for a mechanism to simplify the surgical procedures involved in fastening a rod to the vertebrae of the spine so that the technique can be used more widely.

According to a first aspect of the present invention, there is provided a fastening for attaching a rod to a body part. The fastening can comprise a split ring of a material with a channel for receiving the rod. The fastening can include a formation for receiving a fixing to secure the fastening to the body part. The fastening can further include a closure mechanism operable to close the ring. The material can be sufficiently flexible or pliable to allow the fastening to be closed and/or opened at least once without weakening.

As the fastening can be opened and/or closed at least once, and preferably a plurality of times, without substantially weakening its structural strength, the fastening can be mounted on a rod in situ connected to the body rather than having to be pre-loaded onto the rod. Further, as the material of the fastening provides the required flexibility, the fastening can have a very simple construction resulting in a low profile and also reducing the amount of foreign material implanted in the body.

The fastening can have a regular or irregular, curved or polygonal cross section. In preferred embodiments, the fastening can have a circular, oval, square or rectangular cross section.

The fastening can be used to stabilise any body part with respect to a support rod. Preferably the body part is a spinal body part and more preferably a vertebra.

The formation can provide a part of the closure mechanism. Alternatively, the closure mechanism and anchoring formation can be separate parts. The anchoring formation and closure mechanism can be provide on substantially opposite sides of the fastening.

The closure mechanism can comprises formations of the anchoring formation adjacent either side of the split.

The split ring can have a thinner region of material to provide the required flexibility. The thinner region can have a thickness less than the thickness of at least a further part of the ring or the rest of the ring and can provide a pivot with no separate parts.

The fastening can be a unitary fastening. The fastening can have a single part. This provides a very simple fastening with no separately moving parts and which is easier to fabricate. The fastening can be made of a single band of material.

The closure mechanism can comprises a first projection and a second projection extending from the ring on opposed sides of the slit. The first and second projections can each having an aperture therein for receiving a part of the fixing. The action of the fixing on the projections can cause the ring to clamp about the rod when anchored to the body part.

The apertures can be slots which extend along a longitudinal axis of the ring. This provides some extra freedom in positioning the fastening relative to the body part.

The first and second projections can provide a part of a ball and socket joint. The first and second projections can include formations around each aperture which can co-operate with a part or parts of the fixing to allow the fixing to pivot.

The fastening can include a collar with a bore there through for receiving the rod in use. The collar can be retained in the channel. The collar can be pivotable with respect to the ring. The collar can have a substantially spherical shape. At least a first inner surface of the ring can include a concave region which mates with the outer surface of the collar. Preferably three concave regions are provided in the inner surface of the ring for mating with the outer surface of the collar.

The cross sectional size of the channel can be less than the cross sectional size of the rod. Hence in use, a strong interference fit between the fastening and rod can be achieved when the ring is partially or fully closed about the rod.

The material can be any bio-compatible material having a suitable flexibility that the fastening can be cycled between closed and open configurations at least once or more than once without significantly weakening. The material can be a shape memory alloy. The shape memory alloy can be a nickel-titanium based alloy. Preferably the shape memory alloy is a near equi-atomic nickel-titanium alloy suitable for medical and/or implant use. Most preferably a nickel (54.5%-57.0% by weight)-titanium alloy as specified in ASTM standard F2063.

Other non-metallic flexible materials can be used such as plastics or polymeric materials. For example, a suitable plastics material would be poly-ethylene or poly-acetal. Polymeric materials provide high flexibility but are lower strength than shape memory alloys or metals.

The shape memory alloy can provide the closure mechanism. The shape memory specification can be such that below a temperature the fastening is sufficiently flexible to be manipulated into an open configuration to engage about the rod and above the temperature the fastening can adopt a closed configuration which clamps about the rod. Hence use of the shape memory feature, rather than the pliability of the shape memory alloy, can provide an automatic closure mechanism in which the fastening clamps about the rod as it is heated. Hence no ancillary fixings are required in order to clamp the fastening about the rod.

According to a further aspect of the invention, there is provided a kit of parts for use in a surgical procedure to immobilise vertebrae. The kit can comprise a fastening according to the preceding aspect of the invention and a fixing for securing the fastening to a body part, the fixing having a first formation at a first end for attaching the fixing to a body part and a second formation at a second end, the second formation engaging the anchoring formation of the fastening. The fixing can be operable to close the ring about the rod.*

The fixing can include a screw or a hook. The first formation of the fixing can comprise a screw or hook. The second formation of the fixing can engage with the closure mechanism to clamp the fastening about the rod in use.

According to a further aspect of the invention, there is provided a method for stabilising a vertebra using a split ring fastening, the method can comprise attaching a rod to a body part with a portion of the rod adjacent the vertebra, engaging the fastening about the portion of the rod via a mouth of the fastener, closing the fastening to clamp about the portion of the rod and securing the fastening to a fixing attached to, or to be attached to, the vertebra.

An embodiment of the invention will now be described, by way of example only, and with reference to the accompanying drawings, in which:

FIGS. 1A and 1B respectively show a first version of a fastening according to the invention in open and closed states;

FIG. 2 shows a further version of a fastening according to the invention in an open state;

FIG. 3 shows a further version of a fastening according to the invention in an open state;

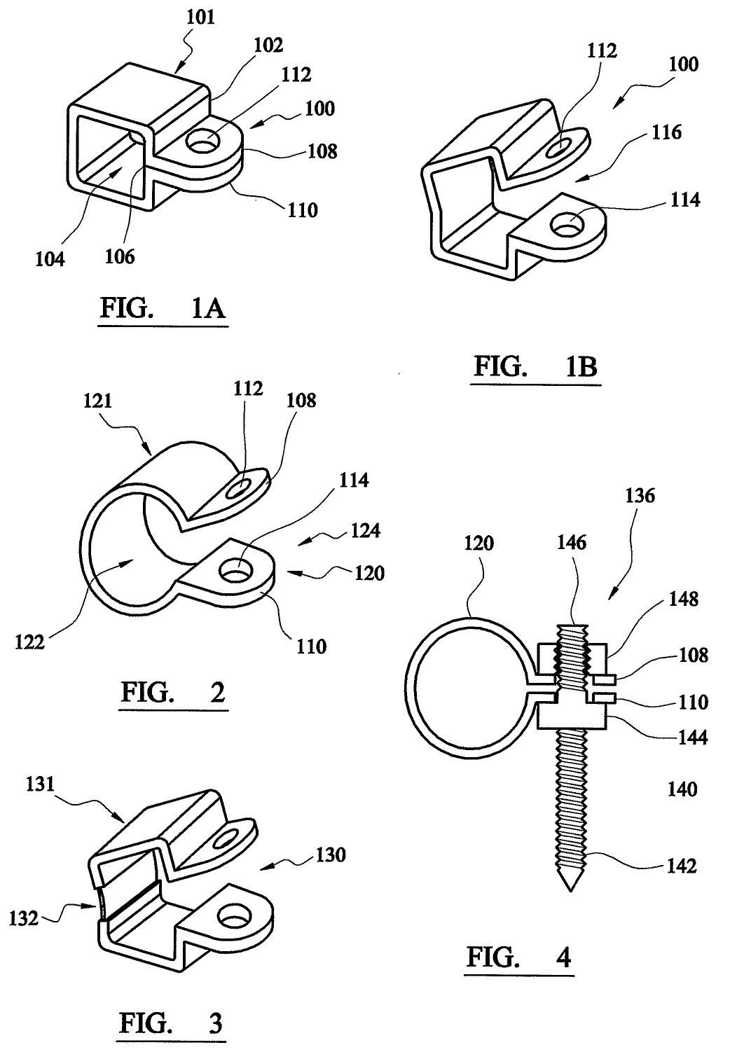

FIG. 4 shows a cross section through an assembly of the fastening of FIG. 3 and a fixing;

FIGS. 5A and 5B respectively show further versions of the fastening of FIGS. 1A and 1B including a pivotable collar;

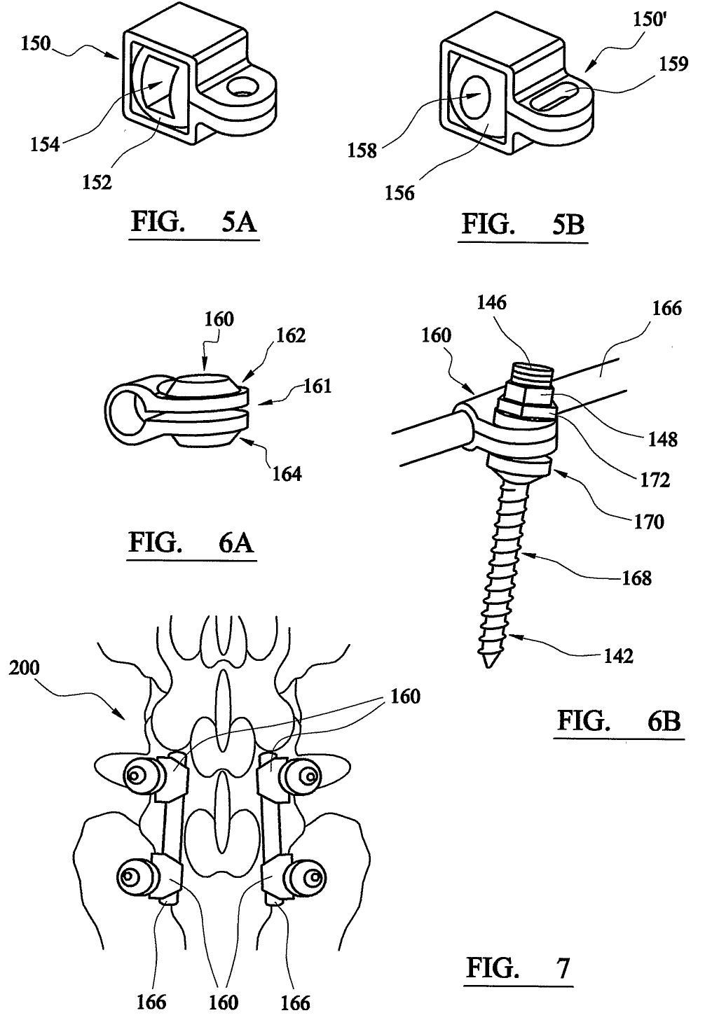

FIGS. 6A and 6B respectively show a further version of a fastening according to the invention in a closed state and mounted on a rod with a fixing;

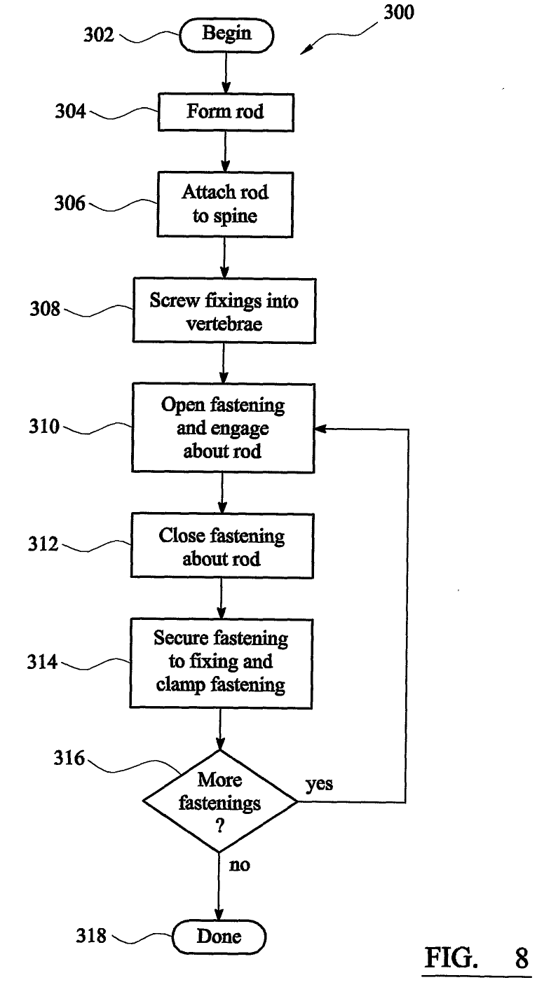

FIG. 7 shows a number of the fastenings of FIG. 6A in use;

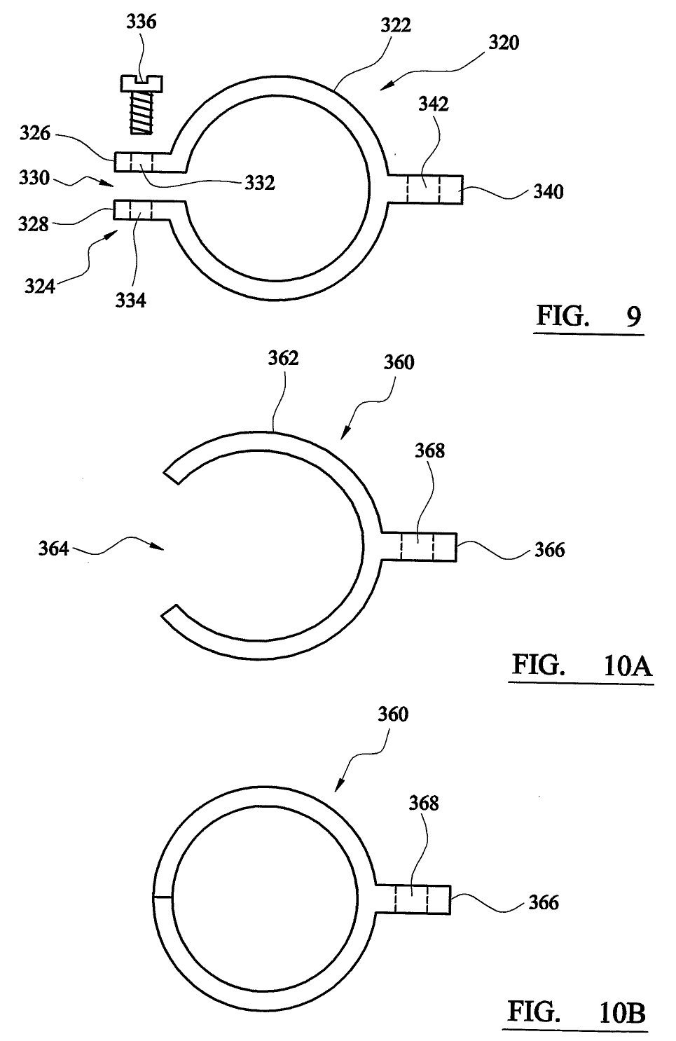

FIG. 8 shows a flow chart illustrating a method of using the fastening according to the invention;

FIG. 9 shows a further version of a fastening according to the invention in a partially open state; and

FIGS. 10A and 10B respectively show a further version of a fastening according to the invention in open and closed states;

Similar items in different Figures share common reference numerals unless indicated otherwise.

With reference to FIGS. 1A and 1B there is shown a fastening 100 according to the present invention in closed and open states respectively. This embodiment of the fastening 100 has a unitary construction, i.e. is a single component, and has a body 101 with a generally split ring shape. The fastening 100 has a substantially square cross-section. The fastener is made of a band of material 102 arranged in a split ring configuration and defining a square channel 104 extending along a longitudinal axis of the fastener.

On either side of the split 106, the body morphs into a first tab 108 and a second tab 110 which projecting from the body. Tabs 108 and 110 provide between them a closure mechanism for clamping the fastener about a rod as will be described in greater detail below. Upper tab 108 includes a countersunk aperture 112 therein. Lower tab 110 also includes an aperture 114 therein in registration with aperture 112. Apertures 112 and 114 in use receive a part of a fitting as will be described in greater detail below.

The whole of fastener 100 is made of shape memory alloy, sometimes, but not exclusively, provided under the trade name Nitinol. Preferably the shape memory alloy meets the requirements of ASTM standard F2063. The super elastic properties of shape memory alloys allows the fastener to be repeatedly cycled between its closed state 100 and its open state 100 by applying a mechanical stress to the fastener and without causing a permanent deformation. The super-elasticity also allows for non-plastic deformations up to ten times those of other implant materials. In the open state shown in FIG. 1B, the fastener has an open mouth 116 at the split in the ring of the body by which the fastening can in use be engaged about a rod as will be described in greater detail below.

The enhanced elastic properties available from shape memory alloys as a result of a transformation between martensite and austenite phases of the alloys make them particularly well suited to use in self-expanding stents. The nature of the superelastic transformations of shape memory alloys is discussed in “Engineering Aspects of Shape Memory Alloys”, T W Duerig et al, on page 370, Butterworth-Heinemann (1990). Subject matter disclosed in that document is incorporated in this specification by this reference to the document. A principal transformation of shape memory alloys involves an initial increase in strain, approximately linearly with stress. This behaviour is reversible, and corresponds to conventional elastic deformation. Subsequent increases in strain are accompanied by little or no increase in stress, over a limited range of strain to the end of the “loading plateau”. The loading plateau stress is defined by the inflection point on the stress/strain graph. Subsequent increases in strain are accompanied by increases in stress. On unloading, there is a decline in stress with reducing strain to the start of the “unloading plateau” evidenced by the existence of an inflection point along which stress changes little with reducing strain. At the end of the unloading plateau, stress reduces with reducing strain. The unloading plateau stress is also defined by the inflection point on the stress/strain graph. Any residual strain after unloading to zero stress is the permanent set of the sample. Characteristics of this deformation, the loading plateau, the unloading plateau, the elastic modulus, the plateau length and the permanent set (defined with respect to a specific total deformation) are established, and are defined in, for example, “Engineering Aspects of Shape Memory Alloys”, on page 376.

Non-linear superelastic properties can be introduced in a shape memory alloy by a process which involves cold working the alloy, for example by a process that involves pressing, swaging or drawing. The cold working step is followed by an annealing step while the component is restrained at a temperature that is sufficiently high to cause dislocations introduced by the cold working to combine and dislocations to align.

The technique for introducing superelastic properties can be varied from that described above. For example, instead of subjecting the alloy to a heat treatment while restrained in the deformed configuration, the alloy could be deformed beyond a particular desired configuration and then heat treated such that there is a thermally induced change in configuration of the kind discussed below, the change taking the configuration towards the particular desired configuration. Introduction of the superelastic properties might also involve annealing at high temperature (for example towards the recrystallisation temperature of the alloy), followed by rapid cooling and then a heat treatment at a lower temperature.

FIG. 2 shows an embodiment of a further fastening 120 according to the present invention. Fastening 120 also has a generally split ring configuration but with a substantially circular cross-section and defining a substantially circular channel 122 therein extending along a longitudinal axis of the fastener. Fastener 120 is shown in an open state in which the fastener has a mouth 124 defined by the opening in the generally ring shaped body 121 of the fastener. Fastener 120 also has a unitary construction and is made from a single band of shape memory alloy, such as those described above.

FIG. 3 shows a further version of a fastener 130 according to the present invention in an open state. Fastener 130 is similar to fastener 100. However, fastener 130 is not made of a shape memory alloy. Rather, fastener 130 has a portion 132 of a wall of the ring shaped body 131 with a thickness narrower than the thickness of the rest of the body. The thin wall portion 132 provides an area of reduced stiffness and provides a mechanical hinge by which the fastener 130 can be opened and closed a number of times without substantially reducing the strength of the fastener. The reduced thickness wall part can be considered a mechanical hinge in that it has no separate relatively moving parts.

As illustrated in FIG. 3, there is a discontinuous change in the thickness of the wall at region 132. However, in other embodiments, there can be a continuous change in the thickness of the wall, i.e. a gradual decrease to a thinner wall section followed by a gradual increase to a thicker wall section corresponding to the thickness of the remainder of the fastener.

Fastener 130 of FIG. 3 can be made from a non-super elastic bio-compatible material, such as titanium. Fastening 130 also has a unitary structure and is made from a single band of titanium.

FIG. 4 shows a cross-sectional view of an assembly 136 comprising a fastener 120 as shown in FIG. 2 and a fixing 140. Fixing 140 includes a lower threaded portion 142 which in use is screwed into a vertebra or other bone body part. A circular flange 144 extends around fixing 140 and a further threaded portion 146 is provided toward an upper end of the fixing. A nut 148 with a thread matching that of the upper part of the fixing is disposed about the upper part 146 of the fixing. The upper part of the fixing 146 passes through apertures 110 and 112 of fastener 120. Fastener 120 is clamped in a closed state by the action of bolt 148 and flange 144 on projecting flaps 108 and 110.

FIGS. 5A and 5B show respective perspective views of two further embodiments of a fastener of the present invention. With reference to FIG. 5A, fastener 150 is substantially similar to fastener 100 as shown in FIG. 1A but includes a collar 152 disposed within the channel defined by the substantially ring shaped body of the fastener. Collar 152 has a substantially spherical shape and has a channel 154 there through with a substantially square cross-sectional shape. In use, channel 154 receives a rod with a square cross section. The inner surfaces of the upper, lower and side walls of the body have concave regions substantially matching the shape of the spherical collar so as to allow the collar to pivot about three mutually perpendicular axes of motion. The concave regions and also retain the collar 152 within the fastener.

FIG. 5B shows a further embodiment 150′ of the fastener similar to that shown in FIG. 5A but in which collar 156 has an aperture 158 with a substantially circular cross-section there through. In use aperture 158 receives a rod with a substantially circular cross section. Also fastening 150′ has apertures in the form of a slot 159 which extend along the longitudinal axis of the fastening thereby allowing the surgeon to anchor the fastening at an appropriate position on a vertebra by providing an extra degree of freedom of movement.

Collars 152 and 156 are made from a bio-compatible material, such as titanium, titanium alloys, stainless steel or ceramic materials. Collars 152, 156 can also be made of bio-compatible plastics and polymeric materials, such as ploy-ethylene and poly-acetal. The surface of the collar and/or the inner surface of the fastener body can include a friction reducing coating, such as a ceramic material or titanium nitride, so as to help improve the pivoting of the collar within the body of the fastener.

FIG. 6A shows a perspective view of a further embodiment of a fastener 160 according to the invention. Fastener 160 is generally similar to fastener 120 shown in FIG. 2. Fastener 160 has a multi-axial closure mechanism providing greater freedom in the positioning of the fastening and its fixing to a bone. The closure mechanism 161 includes a rim 162 surrounding the aperture in the upper part and a further mirror image rim 164 surrounding an aperture in the lower part. The outer surface of rims 162 and 164 generally correspond to the shape of a part of a sphere. The inner walls of the rims 162 and 164 have a generally countersunk shape.

FIG. 6B shows a perspective view of fastener 160 mounted on a section of rod 166 and including a fixing 168, generally similar to fixing 140 shown in FIG. 4. An upper surface of flange 170 has a shape corresponding to a part of a sphere and is shaped to match and engage with outer surface of rim 164. Fastener 168 also includes washer 172 which has an under surface having a shape corresponding to a part of a sphere to match and engage with the outer surface of rim 162. Between them, washer 172, flange 170 and rims 162 and 164 provide a ball and socket type universal joint allowing fixing 168 to pivot and assume any orientation within a conical region extending about an axis passing through the apertures in the closure mechanism.

FIG. 7 shows a plan view 200 of a portion of a spine including vertebra immobilised using the fasteners 160 and fixings 168 illustrating FIGS. 6A and 6B. FIG. 7 is for illustrative purposes only and in practice, rods 166 can extend further along the spine and more vertebra can be attached to the rods 166. A first portion of rod 166 is disposed to the left of the spine and a second portion is disposed to the right of the spine. A lower vertebra is attached to both the rods by a fixing 168 which is screwed into the vertebra and the fastener 160 engages about rod 166 and is secured to the upper part of fixing 168. The adjacent vertebra is similarly secured to the rods by further fasteners 160 which are secured to the upper vertebra by further fixings 168 screwed into the upper vertebra.

A method of use 300 of the fastenings according to the present invention will now be described with reference to a flowchart shown in FIG. 8. Various surgical steps will precede and follow the steps to be described but have been omitted for the sake of clarity. The surgical procedure is begun at 302 and at step 304 the rod or rods can be bent or otherwise shaped into a preferred form to provide the support required to the vertebra or vertebrae to be immobilised by the procedure. At step 306, the rod can be attached to the spine using suitable fixings, such as pedicel screws or hooks at each end of each rod. This anchors the rod to the spine such that the rod is now in situ. At step 308, fixings 168 are screwed into the vertebrae to be supported.

At step 310, if the fastener is already in the closed state then the fastening is opened and the mouth of the fastening is presented to the rod and the fastening is engaged about the rod with the rod extending through the channel within the ring-like body of the fastening. It will be appreciated that if the fastening is provided in an open state, then it is not necessary to open the fastening before presenting it and engaging it about the rod. At step 312 the fastening is closed about the rod and the free end 146 of fixing 168 is passed through the apertures in the closing formation of the fastening. Before securely clamping the fastening about the rod, the fastening can be manipulated by the surgeon to ensure that the vertebra will be supported in the correct position and orientation relative to the rod. Some degree of flexibility of positioning of the fastening is provided by the ball and socket type joint of fixing 168 and/or by the inclusion of the pivotable collar as shown in FIGS. 5A and 5B.

When the surgeon is happy with the positioning of the fastener, the fastener can be clamped in position by screwing down bolt 148 which closes the fastening about the rod thereby clamping the fastening in place. It is beneficial if the inner dimension of the ring is smaller than the corresponding dimension of the rod to ensure a tight interference fit between the fastener and rod when the fastener is clamped about the rod. Once the fastening has been secured to the fixing and the clamp fastened at step 314, then the surgeon can determine if any more fastenings are required at step 316 and if so further fastenings can be added to the rod in situ and clamped in position until all the vertebra have been suitably immobilised. This part of the surgical procedure is then completed at step 318 and further surgical operations can subsequently be carried out.

It will be appreciated that the pliable fastening of the present invention provides a number of benefits. The fastening of the present invention does not require the rod to be pre-loaded prior to being connected to the spine and so it is easier to initially attach the rod to the spine and also to subsequently add further fastenings to the spine as may be required intra-operatively. Further, the fastening of the present invention has a low profile and so causes less disruption to surrounding soft tissues and is easier to accommodate in or around the surrounding body parts. Further, the fastening has a very simple construction and reduces the number of components and amount of foreign material implanted in the patients body. Furthermore, the simple construction of the fastening makes it easier to manufacture initially. Hence the fastening of the present invention enables the use of an otherwise more difficult surgical procedure, has a low profile design, improves the flexibility of the surgical procedure while providing a fastener whose strength is not compromised.

In another embodiment the clamp is made from a shape memory alloy and below the transition temperature the fastener is in the open configuration and above the transition temperature, in the human body, the clamp is in the closed configuration.

FIG. 9 shows a further embodiment of a fastener according to the invention. Fastener 320 is similar to fastener 120 and has a split ring body 322 with a generally circular cross-section defining a channel along a longitudinal axis thereof. A closure mechanism 324 is provided adjacent the split in the ring and includes a first projection 326 and a second projection 328 adjacent either side of the split and providing a mouth 330 to the fastening when in an open state. An aperture 332 is provided in the upper projection 326 and a threaded aperture 334 is provided in the lower projection in registration with the upper aperture. In use, a bolt 336 passes through aperture 332 and is screwed into threaded aperture 334 to close the fastening about a support rod disposed within the body 322 of the fastening.

A member 340 extends from the outer surface of the body 332 of the fastening on the other side of the body and has an aperture 342 therein for receiving a part of a fixing by which the fastening can be secured to the fixing. For example aperture 342 can received free end 146 of fixing 140. Hence in this embodiment, fastening 320 is secured to fixing 140 using element 340 and the support rod is introduced into the fastening by splaying the fastening and clamping the fastening closed about the rod by the action of bolt 336 in closure mechanism 324. Fastening 320 is made of a shape memory alloy to provide the required pliability.

FIGS. 10A and 10B respectively show a further embodiment of a fastening 360 according to the present invention in open and closed configurations. Fastening 360 has a body part 362 in the form of a split ring which has a mouth 364 when the free ends of the split ring are splayed for receiving a rod. A projection 366 extends from an outer surface of body 362 on a generally opposite side to the mouth and has an aperture 368 therein for receiving a fixing for securing the fastening to a vertebra. The split ring body at least is made of a single band of shape memory alloy and the shape memory feature of the alloy is used to provide a closure mechanism.

The properties of shape memory alloys can involve thermally induced changes in configuration in which an article is first deformed from a heat-stable configuration to a heat-unstable configuration while the alloy is in its martensite phase. Subsequent exposure to increased temperature results in a change in configuration from the heat-unstable configuration towards the original heat-stable configuration as the alloy reverts from its martensite phase to its austenite phase.

The shape memory specification is selected such that at a lower temperature, the fastening is pliable and can easily be opened to engage about the rod. Then as the temperature of the fastening is increased in the body, the fastening will change shape into the closed configuration in which the fastening clamps about the rod. Hence, in this embodiment no ancillary fixing or screw is required in order to close the ring about the rod and clamp the rod. The shape memory alloy compositions is selected so as to have a transition temperature, above which the material transforms to clamp closed, in the range of approximately 30 to 37° C. Closure of the fastening during surgery can be stimulated by adding heat to the fastening, e.g. by using warm water or any other suitable heat source, so as to raise its temperature well beyond the body temperature of the patient. Cooling down of the fastening to ambient body temperature has no significant consequences for the clamping force of the fastening.

It is not essential that the fastening 360 closes entirely about the rod such that the free ends of the split ring meet. It is sufficient that the fastening closes sufficiently to clamp securely about the rod to prevent the rod from leaving the fastening via a partially closed mouth in which the free ends do not meet.

It will be appreciated that there are a number of variations in the details of various embodiments of the fasteners, fixings and assemblies thereof and the embodiment shown are by way of illustration only. For example, fixings 140, 168 can include a hook toward a lower part which in use engages about a part of a vertebra rather than a screw. Various features of the different embodiments illustrated can be mixed with features of others of the embodiments illustrated.

Further, some of the steps of the surgical procedure are optional and/or their order can be changed and the operations added to other steps in the procedure or combined. For example fixings can be connected to the vertebra before or after fasteners are connected to the rod. Similarly, all the fasteners can be added to the rod at the same time and/or all the fixings can be screwed into the vertebra during the same step. Alternatively, fixings and fastenings can be attached to the vertebra and rod, on a vertebra by vertebra basis if preferred. Other variations in the method described will be apparent to the person of ordinary skill in the art.

1. A fastening for attaching a rod to a spinal body part, the fastening comprising:
a split ring of a material which defines a channel for receiving the rod;
a formation for receiving a fixing to secure the fastening to the spinal body part; and
a closure mechanism operable to close the ring, wherein the material is sufficiently flexible to allow the fastening to be closed about the rod at least once, without weakening.

2. A fastening as claimed in claim 1, wherein the material is a shape memory alloy.

3. A fastening as claimed in claim 1, wherein the formation is a part of the closure mechanism.

4. A fastening as claimed in claim 3, wherein the closure mechanism comprises formations adjacent either side of the split.

5. A fastening as claimed in claim 1, wherein the split ring has a region of material with a thickness less than the thickness of the remainder of the ring and which provides a mechanical pivot.

6. A fastening as claimed in claim 1, wherein the fastening is a single part.

7. A fastening as claimed in claim 1 wherein the closure mechanism comprises a first projection and a second projection extending from the ring on opposed sides of the slit, the first and second projections each having an aperture therein for receiving a part of the fixing.

8. A fastening as claimed in claim 7, wherein the apertures are slots which extend along a longitudinal axis of the ring.

9. A fastening as claimed in claim 7, wherein the first and second projections include formations around each aperture which can co-operate with a part of the fixing to allow a fixing to pivot with respect to the fastening.

10. A fastening as claimed in claim 1, and further comprising a collar with a bore there through for receiving the rod in use, the collar being retained in the channel and being pivotable with respect to the ring.

11. A fastening as claimed in claim 10, wherein the collar has a substantially spherical shape and at least a first inner surface of the ring includes a concave region which mates with the outer surface of the collar.

12. A fastening as claimed in claim 1, wherein the cross sectional size of the channel is less than the cross sectional size of the rod, such that in use, an interference fit between the fastening and rod can be provided when closing the ring about the rod.

13. A fastening as claimed in claim 12, wherein the shape memory alloy has a shape memory specification such that below a temperature the fastening is sufficiently flexible to adopt an open configuration to engage about the rod and above the temperature the fastening adopts a closed configuration in which the fastening clamps about the rod.

14. A kit of parts for use in a surgical procedure to immobilise vertebrae, the kit comprising:
a fastening having
a split ring of a material which defines a channel for receiving the rod;
a formation for receiving a fixing to secure the fastening to the spinal body part; and
a closure mechanism operable to close the ring, wherein the material is sufficiently flexible to allow the fastening to be closed about the rod at least once, without weakening; and
a fixing for securing the fastening to a body part, the fixing having a first formation at a first end for attaching the fixing to a body part and a second formation at a second end, the second formation engaging the fastening to anchor the fastening to the body part.

15. The kit of claim 14, wherein the fastening includes a second formation at a second end to engage the closure mechanism and be operable to close the ring about the rod.

16. A method for stabilising a vertebra using a split ring fastening, the method comprising:
attaching a rod to a body part with a portion of the rod adjacent the vertebra;
engaging the fastening about the portion of the rod via a mouth of the fastener;
closing the fastening to clamp about the portion of the rod; and
securing the fastening to a fixing for attaching the fastening to the vertebra.

17. (canceled)

18. (canceled)
